(19) United States
(12) Patent Application Publication
(10) Pub. No.: US 2016/0152477 A1
(43) Pub. Date: Jun. 2, 2016

(54) GENERAL METHOD FOR FUNCTIONALIZING CARBON NANOTUBES VIA SOLVENT FREE DIELS-ALDER REACTIONS

(71) Applicant: SHANGHAI PENGXIN ADVANCED MATERIALS TECHNOLOGIES, LTD., Shanghai (CN)

(72) Inventors: Shengxiong XIAO, Jiangsu (CN); Yan LI, Shandong (CN)

(21) Appl. No.: 14/899,365

(22) PCT Filed: Jun. 17, 2013
(86) PCT No.: PCT/CN2013/077314

§ 371 Date: Dec. 17, 2015

(51) Int. Cl. C01B 31/02; C07C 253/30; C07C 255/47; C07C 46/00; C07C 13/64; C07D 209/56; C07D 307/77; C07C 2/86; C07C 13/62; C07D 493/08; C07C 50/22

(52) U.S. Cl. CPC C01B 31/0273; C01B 31/0253; C07D 493/08; C07C 253/30; C07C 255/47; C07C 46/00; C07C 50/22; C07D 209/56; C07D 307/77; C07C 2/86; C07C 13/62; C07C 13/64; C01B 2202/02; C01B 2202/04; C01B 2202/06; C01B 2202/22; C07C 2103/54; C07C 2103/90

(57) ABSTRACT

The present invention provides methods by which carbon nanotubes can be functionalized via Diels-Alder reactions under solvent free conditions. Such methods include reacting carbon nanotubes with Diels-Alder dienes or dienophiles to obtain adducts that includes the diene or dienophile moiety bound to the carbon nanotubes. Functionalized carbon nanotubes and dispersions containing functionalized carbon nanotubes are provided. The present invention provides functionalization methods of carbon nanotubes through gas phase, liquid phase, or solid phase reactions without any solvents other than the reactants. Such processes are also amenable to a wide variety of chemical reactions that use other functionalizing agents. Additionally, such methods are cost effective, easily scalable and can provide for functionalized CNTs in large, industrial-scale quantities.

### FIELD OF THE INVENTION

The present invention relates broadly to carbon nanotubes. More specifically, the invention relates to functionalization of carbon nanotubes via Diels-Alder or hetero Diels-Alder reaction under solvent free conditions, and the dispersions of the functionalized carbon nanotubes. In particular, this application is directed to improved methods of preparing functionalized carbon nanotubes having improved solubility properties and dispersion properties in a solvent or a polymer matrix on industrial scale.

### BACKGROUND OF THE INVENTION

Carbon nanotubes (CNTs, aka fullerene pipes) are nanoscale carbon structures comprising graphene sheets conceptually rolled up on themselves and closed at their ends by fullerene caps. Single-walled carbon nanotubes (SWCNTs) comprise but a single such graphene cylinder, while multi-walled nanotubes (MWCNTs) are made of two or more concentric graphene layers nested one within another in a manner analogous to that of a Russian nesting doll.

Carbon nanotubes possess unique properties making them useful, among others, for the enhancement of electrical, but also thermal conductivity. However, to take advantage of these unique properties, miscibility with or dispensability in a range of hosts is necessary.

There are two major approaches to tackle the challenge: through mechanical mixing and chemical modification (functionalization) of CNTs. Mechanical mixing in the solid state has been used to utilize the carbon nanotubes, but usually does not allow for sufficient mixing at the nanoscale level as van der Waals force based attraction between individual CNT (leading to bundles) is often stronger than attractive forces between CNTs and the host. Surfactants such as, but not limited to, sodium cholate and sodium dodecyl sulfate (SDS), can wrap around individual CNT and break the bundling forces, e.g., leading to debundling, also called exfoliation. However, to achieve this result significant quantities of surfactant are required, which often negatively affect the characteristics of the resulting mixture.

One facile method for the chemical functionalization of the graphene-based structure of the carbon nanofillers is the Diels-Alder (DA) cycloaddition reaction. In previous art, even though it has been proved experimentally that the DA reaction can be performed on carbon nanotubes (CNTs), there are many issues that have to be solved before the methods to be generally applicable.

First of all, in many case, external energy, such as microwaves, has to be applied for the reactions to work. Examples are J. L. Delgado, P. d. l. Cruz, F. Langa, A. Urbina, J. Casado, J. T. L. Navarrete, Chem. Commun. 2004, 1734; [4+2] cycloadditions such as the Diels-Alder cycloaddition of o-quinodimethane with CNTs has also been reported with the assistance of microwave radiation. Similarly, cyclopropanation of malonic acid derivatives could be carried with ultrashort (20 to 80 nm) carbon nanotubes under mild conditions, whereas microwave-assistance was also necessary for longer tubes.

Other DA reactions with CNTs require very critical reaction conditions, such as by using fluorinated CNT starting materials (L. Zhang, J. Yang, C. L. Edwards, L. B. Alemany, V. N. Khabashesku, A. R. Barron, Chem. Commun. 2005, 3265), or by metal complex catalyzed high pressure reactions (C. Menard-Moyon, F. Dumas, E. Doris, C. Mioskowski, J. Am. Chem. Soc. 2006, 128, 14764). The benzocyclobutane based compounds, which are unstable species that has to be produced in situ, have also been used for the DA with CNT (G. Sakellariou, H. Ji, J. W. Mays, N. Hadjichristidis, D. Baskaran, Chem. Mater. 2007, 19, 6370), while this method involves complicated synthetic procedures and unknown side reactions during the DA with CNT may occur.

More recently, DA reactions on multi-walled carbon nanotubes (MWCNTs) have been reported (S. Munirasu, V. Abetz, A. Boschetti-de-Fierro, “Functionalization of Multi-walled Carbon Nanotubes by Diels-Alder Reaction—“Grafting to” Approach”, in Microstructural control in free radical polymerization, G. Schmidt-Naake, Ed., Clausthal, Germany 2008; C.-M. Chang, Y.-L. Liu, Carbon 2009, 47, 3041; Selvaraj Munirasu, Julio Albuerne, Adriana Boschetti-de-Fierro, Volker Abetz, Macromolecular Rapid Communications, 2010, Volume 31, Issue 6, 574-579). However, the DA reactions reported are delicate and vulnerable to temperature and are problematic in the presence of oxygen.

In addition to the issued related to previous art, the greatest limitation of the above mentioned chemical modification of carbon nanotubes method is their very limited solubility in organic solvents or polymer matrixes, which are great obstacles for commercial and industrial applications of nanotubes. Accordingly, there is a need for a technique that will improve the ability to utilize nanotubes without sacrificing their beneficial properties.

The present invention showed great advantages and improvements towards the enhanced dispersion properties of functionalized carbon nanotubes through solvent free Diels-Alder reactions, as listed in the following:

Reactions between carbon nanotubes and a number of Diels-Alder dienes and dienophiles have been reported for the first time. The list of Diels-Alder dienes and dienophiles includes: cyclopentadiene (generated from cracking dicyclopentadiene in situ), anthracene, acrylonitrile, benzoquinone, etc.

Significantly greater degree of functionalization: the concentration of the functional groups on the carbon materials obtained through the above-mentioned methods are generally yielded less than 0.61 mmol/g, while in current invention, the degree of the functional groups could be as high as 4.9 mmol/g, which is about 7 times higher than what's reported in previous art under similar conditions. As a consequence, higher degree of functionalization leads to greater dispersibility in solvents. A high concentration of up to 50 mg/mL of functionalized carbon nanotubes in solvents can be obtained. While in previous art, the concentration of carbon nanotubes in solvents is no greater than 5.0 mg/mL.

Solvent free reaction system has its own advantages. In previous art, either a great amount of solvents (over 1000 fold), or a great excess of the liquid reactants (over 100 fold) was used to carry out the reactions as illustrated in Selvaraj Munirasu, Julio Albuerne, Adriana Boschetti-de-Fierro, Volker Abetz, Macromolecular Rapid Communications, 2010, Volume 31, Issue 6, 574-579. Large amount of solvent and large excess of liquid reactants brings quite a great deal of issues such as filtration, distillation, impurities, cost, and environmental pollution, etc.

No external energy, such as microwave, is needed. Which in return reduces the complications of unwanted side reactions.

In one embodiment, active species cyclopentadiene is generated in situ. Due to the high reactivity of this species, the reaction was more efficient. And the product could be easily purified by sublimation of the volatile reactant.

### SUMMARY OF THE INVENTION

A new inexpensive and scalable functionalization method for carbon nanotubes is provided. In one aspect, the method includes heating carbon nanotubes with a Diels-Alder diene or dienophile compound without additional solvents. The method is performed under conditions sufficient to obtain an adduct comprising the Diels-Alder diene or dienophile moiety bound to the carbon nanotube.

In one aspect, dienes or dienophiles were added by Diels-Alder reactions to carbon nanotubes to provide highly functionalized carbon nanotubes. In certain embodiments, a [4+2] Diels-Alder reaction is conducted using the reactant as the solvent, in the range of 0.1˜10 molar ratio to CNTs, i.e. furan and its derivatives, cyclopentadiene or dicyclopentadiene, furfural, acrylonitrile, industrial scale anthracene oil. In other embodiment, solid-state bulk dienes or dienophiles, such as benzoquinone, N-phenylmaleimide, and maleic anhydride, were reacted with CNTs without solvent. A purification procedure based on the sublimation of excess dienes or dienophiles has also been demonstrated.

In some embodiments, the Diels-Alder diene or dienophile compound is an optionally substituted diene or dienophile compound. In other embodiments, the optionally substituted diene or dienophile compound includes one or more functional groups selected from the group consisting of alkyl, C3-C12 alkenyl, alcohol, halogen, metal halide, carboxylic acid, ester, ether, polyethyleneglycol, —NH₂, NHR, —CONH, —SO₃H, ketone, aldehyde, epoxyl, optionally substituted phenyl, optionally substituted benzyl, and mixtures thereof, or where the optionally substituted Diels-Alder diene or dienophile compound is fused with one or more additional aromatic rings. In these embodiments, where R is C1-C12 alkyl, C3-C12 alkenyl, halogen, or a mixture thereof. In further embodiments, the polycyclic aromatic hydrocarbon includes optionally substituted naphthalene, phenanthrene, or anthracene, pentacene.

In some embodiments, such Diels-Alder diene or dienophile species can also be generated in situ, such as cyclopentadiene generated from dicyclopentadiene by heating.

In some embodiments, carbon nanotubes are generally selected from the group consisting of single-wall carbon nanotubes, double-walled carbon nanotubes, multi-walled carbon nanotubes, small diameter carbon nanotubes, and combinations thereof, preferably multi-walled carbon nanotubes.

In some embodiments, the heating step of the method of preparing functionalized carbon nanotubes includes mixing the carbon nanotubes with solid dienes or dienophiles thoroughly before heating to the desired temperature. For example, in some embodiments, sealed tube reaction is provided where the reactants (Diels-Alder dienes or dienophiles) are solid at room temperature.

In some embodiments, the heating step of the method of preparing functionalized carbon nanotubes includes heating the carbon nanotube in the liquid dienes or dienophiles. For example, in some embodiments, sealed tube reaction is provided where the reactants (Diels-Alder diene or dienophile) are liquid at room temperature. In other embodiments, melting is provided where at least one of the reactants is solid at room temperature.

In another aspect, the functionalized carbon nanotubes products are purified by filtering, washing, extraction, drying, resuspending, and combinations thereof, and/or vacuum sublimation to get rid of the excess Diels-Alder diene or dienophiles. In some embodiments, the extraction is solid-liquid extraction (e.g., Soxhlet extraction), while in other embodiments, the extraction is liquid-liquid extraction.

In a further aspect, a method of preparing the stable carbon nanotube dispersion described above is disclosed. In some embodiments, the stable carbon nanotube dispersion is prepared by sonication, and/or stirring of the functionalized carbon nanotubes in a variety of aqueous or organic solvents, polar or nonpolar solvents. In some embodiments, the solvent includes dimethylacetomide (DMA) or N-methyl-2-pyrrolidone (NMP), chloroform, 1,1,2,2-tetrachloroethane, water, tetrahydrofuran (THF), propylene glycol methyl ether acetate (PGMEA), alcohol (e.g., methanol and ethanol), hexane, benzene, toluene, xylenes, chlorobenzene, or mixtures thereof.

In another aspect, a stable carbon nanotube dispersion is provided. The dispersion includes a population of functionalized carbon nanotubes and a solvent. In some embodiments, the functionalized carbon nanotube includes a Diels-Alder diene or dienophile moiety. In some embodiments, the solvent of the dispersion includes DMA or NMP, chloroform, 1,1,2,2-tetrachloroethane, water, THF, PGMEA, alcohol (e.g., methanol and ethanol), hexane, benzene, toluene, xylenes, chlorobenzene, or mixtures thereof.

In some embodiments, the disclosed stable carbon nanotube dispersions have a carbon nanotube content greater than 20 mg/mL. In other embodiments, the carbon nanotube dispersions have a carbon nanotube content greater than 50 mg/mL.

In one aspect, a method of recovering the conductivity of carbon nanotube is provided. The method includes (a) reacting carbon nanotubes with an optionally substituted Diels-Alder diene or dienophile-based compound under conditions sufficient to produce a plurality of Diels-Alder diene or dienophile -carbon nanotube adducts;(b) purifying the Diels-Alder diene or dienophile-carbon nanotube adducts; (c) heating the Diels-Alder diene or dienophile-carbon nanotube adduct under inert conditions to fulfill a retro Diels-Alder reaction to recover the electrical conductivity. In some embodiments, the electrical conductivity is increased by at least 15%, by at least 24%, or by at least about 43% comparing to the functionalized carbon nanotubes.

The foregoing has outlined rather broadly the features of the present invention in order that the detailed description of the invention that follows may be better understood. Additional features and advantages of the invention will be described hereinafter, which form the subject of the claims of the invention.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1. SEM image of as received multi-walled CNTs.

FIG. 2. SEM image of modified multi-walled CNTs after Diels-Alder reactions with cyclopentadiene.

FIG. 3. TGA of CNTs modified with maleic anhydride.

FIG. 4. TGA of CNTs modified with cyclopentadiene.

FIG. 5. XPS spectrum of CNTs modified with maleic anhydride.

### DETAILED DESCRIPTION OF THE INVENTION

The preparation of functionalized carbon nanotubes is described. Purification methods for the functionalized carbon nanotubes are also described. Dispersions of the disclosed functionalized nanotubes are also described, as well as methods for preparing such dispersions. Method to recover the conductivity of the functionalized carbon nanotubes is also described.

In one embodiment, a method of preparing functionalized carbon nanotubes includes heating carbon nanotubes with a Diels-Alder diene or dienophile is described. For example, in one embodiment, a Diels-Alder reaction results in [4+2] addition product of the Diels-Alder diene or dienophile-based reactants with the nanotubes.

A general reaction scheme for reaction of a carbon nanotube with Diels-Alder diene or an optionally substituted Diels-Alder diene according to one embodiment is shown in Scheme 1. The exemplary dienes include cyclopentadiene (generated in situ from dicyclopentadiene by heating), anthracene oil, or furfural. In Scheme 1, no solvent is used. It has been discovered that the disclosed methods successfully achieve functionalization of carbon nanotubes without the addition of externally applied energy in addition to heat (e.g., microwaves, UV irradiation or ultrasound). The method described here can be carried out in any laboratory, is easy to scale and does not require any complex or expensive equipment.

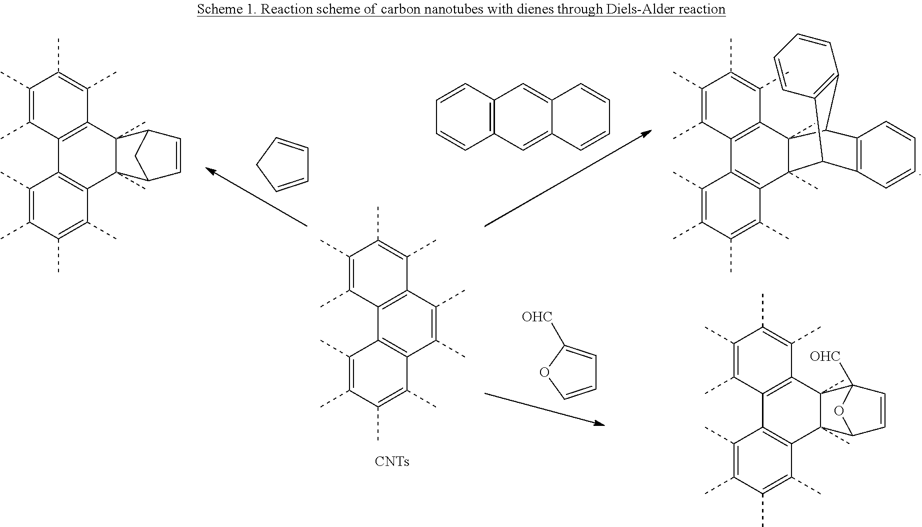

A general reaction scheme for reaction of a carbon nanotube with Diels-Alder dienophile or an optionally substituted Diels-Alder dienophile according to one embodiment is shown in Scheme 2. The exemplary dienophiles include acrylonitrile, benzoquinone, N-phenylmaleimide, and maleic anhydride. In Scheme 2, no solvent is used. It has been discovered that the disclosed methods successfully achieve functionalization of carbon nanotubes without the addition of externally applied energy in addition to heat (e.g., microwaves, UV irradiation or ultrasound). The method described here can be carried out in any laboratory, is easy to scale and does not require any complex or expensive equipment.

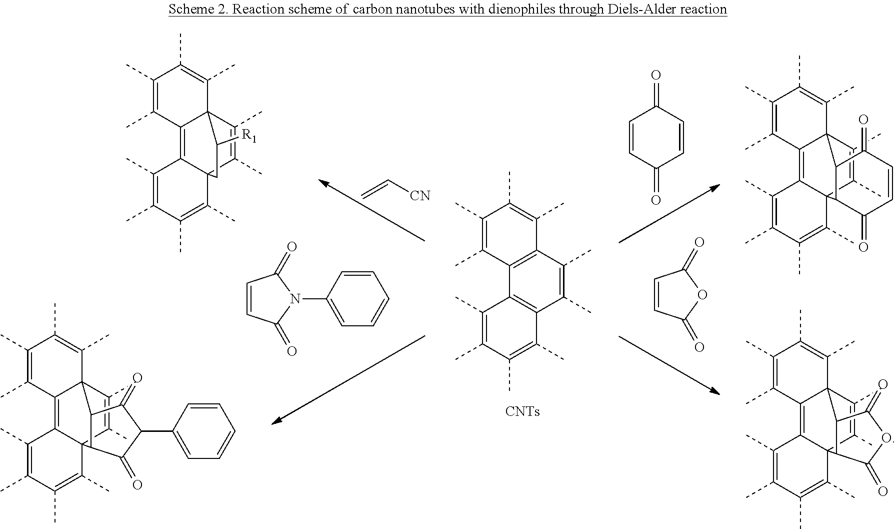

In some embodiments, the reactant is a functionalized Diels-Alder diene or dienophile. In some embodiments, the functionalized Diels-Alder diene or dienophiles incorporate chemical functional groups (e.g., chemical moieties) covalently bound to the Diels-Alder diene or dienophile group. For example, in some nonlimiting embodiments, the Diels-Alder diene or dienophile group includes one chemical function group covalently bound to the Diels-Alder diene or dienophile group. In other nonlimiting embodiments, the Diels-Alder diene or dienophile group includes two chemical function group covalently bound to the Diels-Alder diene or dienophile group. In yet another nonlimiting embodiment, the Diels-Alder diene or dienophile group includes three chemical function group covalently bound to the Diels-Alder diene or dienophile group. In still further nonlimiting embodiments, the Diels-Alder diene or dienophile group includes four chemical function group covalently bound to the six-membered ring of the Diels-Alder diene or dienophile group (i.e., it is fully saturated with functional groups).

Exemplary functionalized Diels-Alder diene or dienophiles include, without limitation, Diels-Alder diene or dienophile that has been modified to include one or more of: C₁-Cₙ alkyl, C₃-C₁₂ alkenyl, halogen (e.g., bromine, chlorine or fluorine), metal halides (e.g., magnesium halide), alcohol (e.g., —ROH), carboxylic acid (e.g., —COOH), ester (e.g., —COOR), ether (e.g., —OR), polyethyleneglycol, —NH₂, NHR, —CONH, —SO₃H, ketones (e.g., —COR), aldehydes (e.g., —CHO), phenyl, benzyl (e.g., —CH₂—C₆H₅), phenyl or benzyl bearing any of the other functionalized groups listed before or combinations between the previous functionalities. In some embodiments, R is C₁-Cₙ alkyl, C₃-C₁₂ alkenyl, or halogen (e.g., bromine, chlorine or fluorine). In some nonlimiting embodiments, the R or functional group is C₁-Cₙ alkyl (e.g., one or more of methyl, ethyl, propyl, isopropyl). In other nonlimiting embodiments, R or the functional group is C₁-C₆ alkyl. In further nonlimiting embodiments, R or the functional group is C₃-C₈ alkyl.

In other embodiments, the reactant is a Diels-Alder diene or dienophile fused with one or more additional aromatic rings. Nonlimiting examples of such additional aromatic rings include 5-8-atom optionally substituted aromatic rings. In another embodiment, the Diels-Alder diene or dienophile is fused with a non-aromatic saturated or partially unsaturated 5-8 atom unsubstituted or substituted ring. In a further embodiment, the Diels-Alder diene or dienophile is fused with a six-membered aromatic ring. Exemplary optional substitutions include, without limitation, C₁-C₁₂ alkyl, C₃-C₁₂ alkenyl, halogen (e.g., bromine, chlorine or fluorine), metal halides (e.g., magnesium halide), C₁-C₁₂ alcohol (e.g., —ROH), carboxylic acid (e.g., —COOH), ester (e.g., —COOR), ether (e.g., —OR), polyethyleneglycol, —NH₂, NHR, —CONH, —SO₃H, ketone (e.g., —COR), aldehyde (e.g., —CHO), phenyl, benzyl (e.g., —CH₂—C₆H₅, phenyl or benzyl bearing any of the other functionalized groups listed before or combinations between the previous functionalities). In some embodiments, R is C₁-Cₙ alkyl, C₃-C₁₂ alkenyl, or halogen (e.g., bromine, chlorine or fluorine).

As disclosed herein, the capability of functionalized carbon nanotubes to be dispersed in various solvents such as water, alcohol (e.g. methanol or ethanol), DMA, NMP, THF, and PGMEA has been demonstrated successfully. Extension to other solvents will be enabled by the attachment of additional functional groups to the reactant (used for the functionalization) prior to reaction. Solubilization of the functionalized carbon nanotube products in a range of solvents can be achieved by choosing suitable functional groups to be attached, taking into account, e.g., the degree of polarity of the both the solvent and attached functionalities. For instance, functionalization of the Diels-Alder diene or dienophile with alkyl groups (CₙH₂ₙ₋₁) or alkenyl (CₙH₂ₙ₋₁) with n>6 will enhance the solubility in aliphatic solvents such as hexane. In other embodiments, attachment of Diels-Alder diene or dienophile bearing aromatic groups such as phenyl, naphthyl, anthracyl, phenantryl, pyrenyl or other substituted or unsubstituted polycyclic aromatic hydrocarbons leads to increased solubility in aromatic solvents such as benzene, toluene, xylenes, or chlorobenzene. Polyethylene glycol attached to the Diels-Alder diene or dienophile will allow for solubility in polar solvents, for instance water and methanol. Solubility will increase with the length of the polyethylene glycol chain.

In certain embodiments, the Diels-Alder diene or dienophile and the carbon nanotubes are heated for a period of time sufficient to achieve functionalization of the carbon nanotubes. In some nonlimiting embodiments, the reactants are heated for a period of about 12 to about 24 hours. For example, in some embodiments, the reactants are heated for at least about 12, at least about 16, at least about 20, or at least about 24 hours. The extent of the functionalization can be controlled by the duration of the heating. For example, refluxing less than 12 hours leads to a reduced density of the functionalization on the carbon nanotube surface. Diels-Alder diene or dienophile (or functionalized Diels-Alder diene or dienophile)—carbon nanotubes ratios can range for 0.1:1 to 10:1. Reduced Diels-Alder diene or dienophile (or functionalized Diels-Alder diene or dienophile) concentrations will lead to a reduced concentration of the attachment on the CNT surface and longer reaction times will be required.

Functionalization of the carbon nanotubes is assessed by spectroscopy, thermogravimetric analysis (TGA), microscopy (e.g., scanning electron microscopy (SEM) or transmission electron microscopy (TEM), or other suitable methods. Spectroscopic methods include, without limitation, X-ray Photoelectron spectroscopy (XPS), Raman, infrared, and UV-Vis.

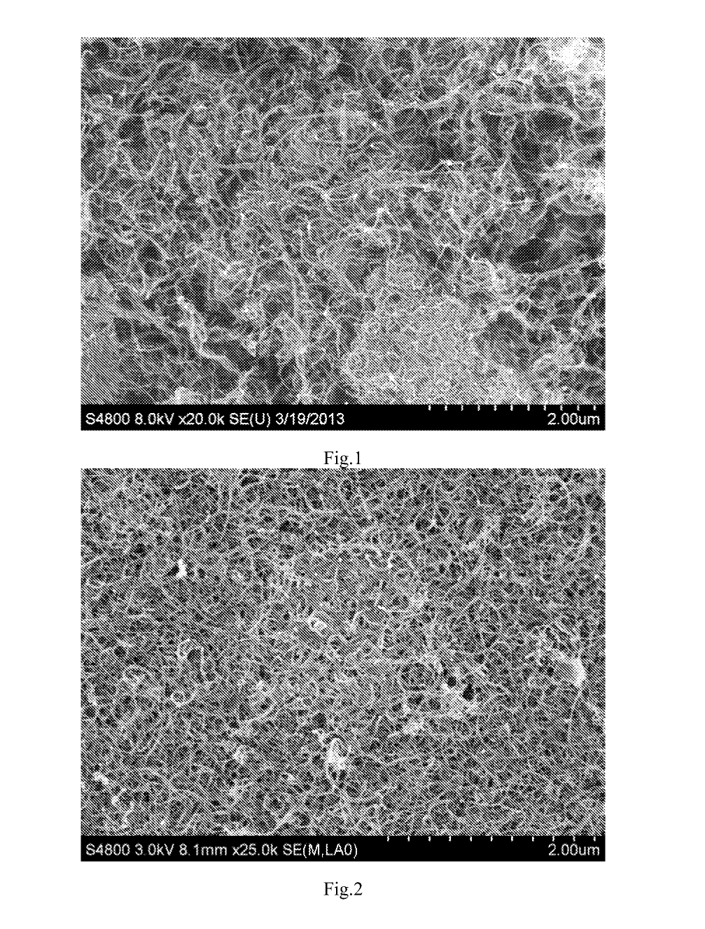

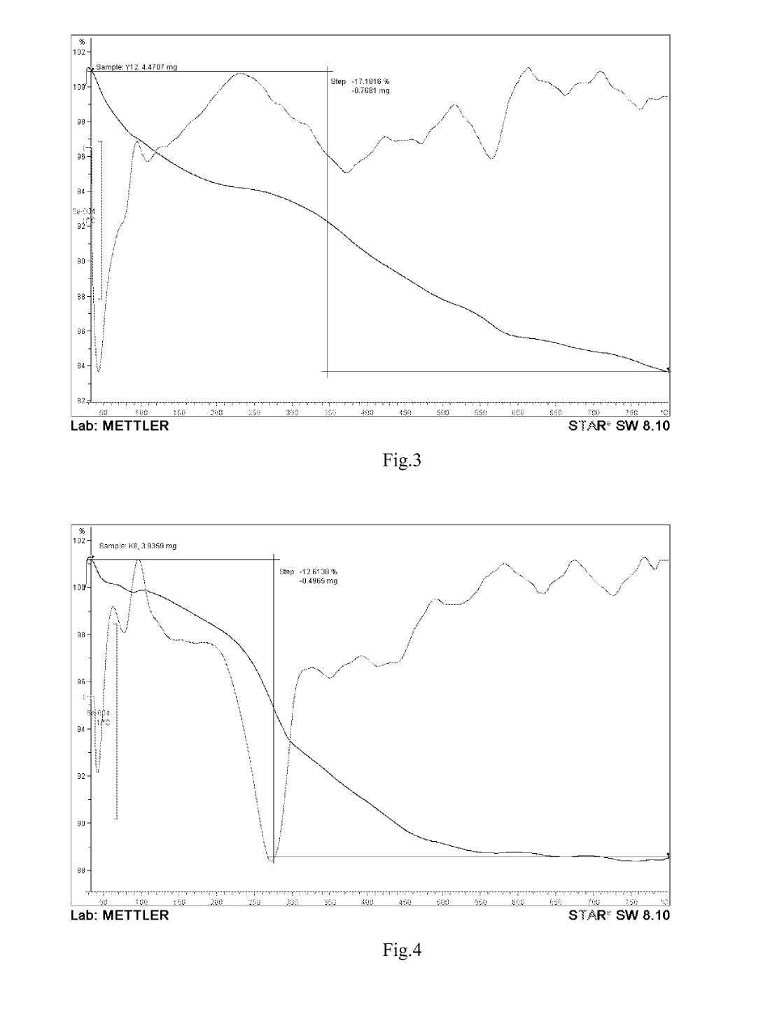

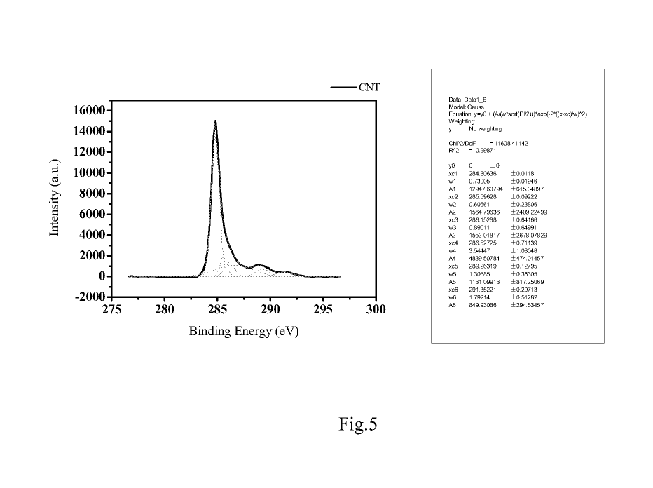

For example, in one embodiment, functionalization is evaluated via XPS, as shown in FIG. 5. The presence of strong the C═O, C—O and —OH signals is indicative of functionalization. In some embodiments, TGA is performed in an inert gas atmosphere. In TGA, functionalized nanotubes exhibit more weight loss than non-functionalized nanotubes, as shown in FIG. 3, and FIG. 4. In other embodiments, surface roughness of the carbon nanotubes, as shown by SEM in FIG. 1, and FIG. 2, is indicative of functionalization. The unfunctionalized carbon nanotubes shown in FIG. 1 showed a great deal of bundles, while after functionalization, the carbon nanotubes are much more uniformly dispersed, as shown in FIG. 2.

It has been found that the functionalized carbon nanotubes prepared by the disclosed methods exhibit superior dispersibility (e.g., improved solubility) in a range of solvents.

Exemplary solvents include, without limitation, NMP, DMA, propylene-glycol-methyl-ether-acetate (PGMEA), ethanol, tetrahydrofuran (THF), o-dichlorobenzene (ODCB), heptane, hexane, and dichloromethane (DCM). For example, in some embodiments, the functionalized nanotubes are dispersed in solvent at a concentration of about 0.1 mg/mL to about 10 mg/mL. In some embodiments, the functionalized nanotubes are dispersed in solvent at a concentration of about 0.2 mg/mL to about 10 mg/mL or about 0.2 mg/mL to about 20 mg/mL. In still further embodiments, the functionalized nanotubes are dispersed in solvent at concentrations of at least about 50 mg/mL or greater than about 50 mg/mL.

Dispersions of carbon nanotubes are prepared by sonicating a mixture of nanotubes in solvent for a specified period of time. In some embodiments, the mixture is sonicated for 15 minutes, 20 minutes, 45 minutes, or about 60 minutes. In other embodiments, the mixture is sonicated for at least about 1 hour.

In some embodiments, dispersions are allowed to stand for about 2 days, about 3 days, about 4 days, or about 5 days. In other embodiments dispersions are allowed to stand for at least about 2 days, about 3 days, about 4 days, about 5 days, or for a month. Phase separation, or observation of precipitation of the carbon nanotubes is indicative of instability in the materials. Dispersion stability is also assessed by subjecting the well-dispersed material (e.g., that has been subjected to sonication) to centrifugation. In some embodiments, solubility is confirmed by ability to withstand centrifugation (e.g., the material is considered soluble when it does not phase separate during centrifugation). Visual observation of precipitation or phase separation is indicative of an instable or insoluble dispersion. In some embodiment, the dispersion is centrifuged at about 1,000, about 2,000, about 5,000, about 7,500, or about 10,000 rpm. In some embodiments, the dispersions are centrifuged for about 15 minutes, about 20 minutes, about 30 minutes, about 45 minutes or about 60 minutes. In other embodiments, the dispersions are centrifuged for at least about 30 minutes or at least about 60 minutes.

In one embodiment, functionalization of the carbon nanotubes is reversed by heating, which fulfills a retro Diels-Alder reaction. For example, in some embodiments, de-functionalization is accomplished by application of heat to the applied nanotubes. In one specific embodiment, functionalized carbon nanotubes are heated in bulk. Once the nanotubes is purified and dried, it is heated to a temperature that can range from about 40° C. to about 400° C., at a pressure ranging from about 10⁻⁶ torr to about 1 atmosphere. In one embodiment, the nanotubes are heated to greater than about 120° C., at atmospheric pressure. In some embodiments, the applied material is heated to a temperature about the boiling temperature of the functionalization reactant (e.g., Diels-Alder diene or dienophile). It is theorized that this heating with or without pressure reverses the functionalization of the nanotubes by undergoing a retro Diels-Alder reaction, thereby restoring the desired high electrical conductivity of the nanotube. As shown in Table 1, the electrical conductivity of functionalized nanotubes decreased about 2˜5 folds comparing to the as received carbon nanotubes, which has a bulk electrical resistance of 1050 uΩ·m), while after annealing, the conductivity increased about 15% to 43% comparing to the functionalized carbon nanotubes. If defunctionalization is conducted in an inert atmosphere, annealing leads to further improvement of the conductivity. In some embodiments, annealing is achieved by heating to temperatures of >about 400° C.

### EXAMPLES

The weight loss of the functionalized carbon nanotubes is about 11.5% as indicated by TGA spectrum performed under inert atmosphere, as shown in FIG. 3. This is transformed into a concentration of functional groups of about 1.7 mmol/g. The bulk resistance of functionalized nanotubes is 3449 uΩ·m, while after annealing, the bulk resistance reduced to 2919 uΩ·m. The detailed data are listed in Table 1.

The weight loss of the functionalized carbon nanotubes is about 30.2% as indicated by TGA spectrum performed under inert atmosphere. This is transformed into a concentration of functional groups of about 1.7 mmol/g. The bulk resistance of functionalized nanotubes is 2097 uΩ·m, while after annealing, the bulk resistance reduced to 1537 uΩ·m. The detailed data are listed in Table 1.

The weight loss of the functionalized carbon nanotubes is about 15.4% as indicated by TGA spectrum performed under inert atmosphere. This is transformed into a concentration of functional groups of about 1.6 mmol/g. The bulk resistance of functionalized nanotubes is 4293 uΩ·m, while after annealing, the bulk resistance reduced to 3544 uΩ·m. The detailed data are listed in Table 1.

The weight loss of the functionalized carbon nanotubes is about 26.1% as indicated by TGA spectrum performed under inert atmosphere. This is transformed into a concentration of functional groups of about 4.9 mmol/g. The bulk resistance of functionalized nanotubes is 5297 uΩ·m, while after annealing, the bulk resistance reduced to 3066 uΩ·m. The detailed data are listed in Table 1.

The weight loss of the functionalized carbon nanotubes is about 15.7% as indicated by TGA spectrum performed under inert atmosphere. This is transformed into a concentration of functional groups of about 1.5 mmol/g. The bulk resistance of functionalized nanotubes is 4593 uΩ·m, while after annealing, the bulk resistance reduced to 3683 uΩ·m. The detailed data are listed in Table 1.

The weight loss of the functionalized carbon nanotubes is about 31.1% as indicated by TGA spectrum performed under inert atmosphere. This is transformed into a concentration of functional groups of about 1.8 mmol/g. The bulk resistance of functionalized nanotubes is 3724 uΩ·m, while after annealing, the bulk resistance reduced to 3055 uΩ·m. The detailed data are listed in Table 1.

The weight loss of the functionalized carbon nanotubes is about 14.3% as indicated by TGA spectrum performed under inert atmosphere. This is transformed into a concentration of functional groups of about 1.5 mmol/g. The bulk resistance of functionalized nanotubes is 3102 uΩ·m, while after annealing, the bulk resistance reduced to 2342 uΩ·m. The detailed data are listed in Table 1.

TABLE 1
Reactions between CNTs and various Diels-Alder dienes and dienophiles.
Conditions: 1.5 g of multi-walled CNTs was reacted at 200° C. (Note:
the electrical resistance of carbon nanotubes was measured in bulk. And
the resistance of the starting carbon nanotubes is around 1050 uΩ · m)

| | | Preferred | | | Resistance | Weight | |
|---|---|---|---|---|---|---|---|
| Entry | | CNTs:re- | Time | Resistance | after | loss | X |
| No. | Reactant | actant Ratio | (h) | (uΩ · m) | annealing | (%) | (mmol/g) |
| 1 | Dicyclopentadiene | 1:1 | 12 | 3449 | 2919 | 11.5 | 1.7 |
| 2 | Anthracene | 1:5 | 14 | 2097 | 1537 | 30.2 | 1.7 |
| 3 | Furfural | 1:5 | 12 | 4293 | 3544 | 15.4 | 1.6 |
| 4 | Acrylonitrile | 1:1 | 12 | 5297 | 3006 | 26.1 | 4.9 |
| 5 | Benzoquinone | 1:1 | 12 | 4593 | 3683 | 15.7 | 1.5 |
| 6 | N-phenylmaleimide | 1:3 | 12 | 3742 | 3035 | 31.1 | 1.8 |
| 7 | Maleic anhydride | 1:1 | 20 | 3102 | 2342 | 14.3 | 1.5 |

Upon review of the description and embodiments of the present invention, those skilled in the art will understand that modifications and equivalent substitutions may be performed in carrying out the invention without departing from the essence of the invention. Thus, the invention is not meant to be limiting by the embodiments described explicitly above, and is limited only by the claims which follow.

1. A method of preparing functionalized carbon nanotubes, comprising:
reacting carbon nanotubes with a diene or dienophile compound through Diels-Alder reaction without additional solvent to obtain an adduct comprising functional moiety covalently bound to the carbon nanotubes.

2. The method of claim 1, wherein the diene is at least one of cyclopentadiene, dicyclopentadiene, furan, butadiene, anthracene, tetracene, and pentacene.

3. The method of claim 1, wherein the dienophile is at least one of maleic anhydride, maleic imide, benzoquinone, alkyne, alkene.

4. The method of claim 1, wherein the Diels-Alder diene or dienophile compound could be generated in situ.

5. The method of claim 1, wherein the Diels-Alder diene or dienophile compound could be optionally substituted.

6. The method of claim 5, wherein the optionally substituted Diels-Alder diene or dienophile-based compound comprises one or more functional groups selected from the group consisting of alkyl, C3-C12 alkenyl, alcohol, halogen, metal halide, carboxylic acid, ester, ether, polyethyleneglycol, —NH₂, NHR, —CONH, —SO₃H, ketone, aldehyde, epoxyl, optionally substituted phenyl, optionally substituted benzyl, and mixtures thereof, or where the optionally substituted Diels-Alder diene or dienophile compound is fused with one or more additional aromatic rings.

7. The method of claim 6, wherein at least one of the one or more additional aromatic rings comprise optionally substituted benzene, naphthalene, phenanthrene, and anthracene.

8. A functionalized carbon nanotube comprising:
a carbon nanotube; and
at least one Diels-Alder diene or dienophile-based functional group covalently bound to the carbon nanotube.

9. The functionalized carbon nanotube of claim 8, wherein the at least one Diels-Alder diene or dienophile-based functional group is optionally substituted with up to four chemical moieties covalently bound to the Diels-Alder diene or dienophile -based functional group.

10. The functionalized carbon nanotube of claim 8, wherein chemical moieties independently comprise at least one of alkyl, C3-C 12 alkenyl, alcohol, halogen, metal halide, carboxylic acid, ester, ether, polyethyleneglycol, —NH₂, NHR, —CONH, —SO₃H, ketone, aldehyde, epoxyl, optionally substituted phenyl, optionally substituted benzyl, and mixtures thereof.

11. The functionalized carbon nanotube of claim 8, wherein the functionalized carbon nanotube has a degree of functionalization of 1.5 mmol/g or more.

12. The functionalized carbon nanotube of claim 8, wherein the functionalized carbon nanotube is produced by heating the a mixture of carbon nanotubes and the Diels-Alder diene or dienophile in the range of 0.1 to 10 molar ratio.

13. The functionalized carbon nanotube of claim 12, wherein reaction temperatures are between 100 to 300° C.

14. The functionalized carbon nanotube of claim 8, wherein the carbon nanotube comprises single-wall carbon nanotubes, double-walled carbon nanotubes, multi-walled carbon nanotubes, small diameter carbon nanotubes, and combinations thereof.

15. The functionalized carbon nanotube of claim 12, wherein the carbon nanotube is preferably a multi-walled carbon nanotube.

16. A method of preparing the stable carbon nanotube dispersion of claim 8, comprising:
providing a population of the functionalized carbon nanotubes; and
dispersing the population of carbon nanotubes in a solvent comprising at least one of DMA or NMP, chloroform, 1,1,2,2-tetrachloroethane, water, THF, PGMEA, alcohol (e.g., methanol and ethanol), hexane, benzene, toluene, xylenes, chlorobenzene, and mixtures thereof.

17. The stable carbon nanotube dispersion of claim 16, wherein the carbon nanotube content is greater than 10 mg/mL.

18. The carbon nanotube dispersion of claim 16, wherein the carbon nanotube content is greater than 50 mg/mL.

19. A method of annealing the functionalized carbon nanotubes in claim 8 to recover its conductivity, which comprises:
reacting a plurality of carbon nanotubes with an optionally substituted Diels-Alder diene or dienophile -based compound under conditions sufficient to produce a plurality of Diels-Alder diene or dienophile -carbon nanotube adducts;
purifying the plurality of Diels-Alder diene or dienophile-carbon nanotube adducts by washing, filtration, sublimation of unreacted diene or dienophile; and
heating the Diels-Alder diene or dienophile-carbon nanotube adduct under inert conditions up to 600° C. to recover the electrical conductivity by at least about 15%, 20%, or 43%.
